(19) United States
(12) Patent Application Publication
(10) Pub. No.: US 2018/0216981 A1
(43) Pub. Date: Aug. 2, 2018

(54) COMPLEX TOOL FOR WELL MONITORING

(71) Applicant: SCHLUMBERGER TECHNOLOGY CORPORATION, Sugar Land, TX (US)

(72) Inventors: Valery Vasilievich Shako, Moscow (RU); Dmitrii Evgenievich Miklashevskiy, Moscow (RU); Sergey Sergeevich Safonov, Moscow (RU)

(21) Appl. No.: 15/640,690

(22) Filed: Jul. 3, 2017

(30) Foreign Application Priority Data
Jul. 1, 2016 (RU) 2016126377

(51) Int. Cl. G01F 1/684; G01K 13/02; G01N 33/28

(52) U.S. Cl. CPC G01F 1/684; G01N 33/2823; G01K 13/02

(57) ABSTRACT

The complex tool for well monitoring comprises a cylindrical housing and at least two lever centralizers aligning the tool along a well axis. Each centralizer has at least three levers, as well as at least one fluid flow temperature sensor, at least one phase composition sensor and at least one thermal flow velocity sensor, all sensors are located on an axis of the tool. The tool also comprises at least three groups of sensors distributed around a perimeter of the wellbore when the levers of at least one centralizer are being opened. Each group of the sensors comprises at least a fluid flow temperature sensor, a fluid phase composition sensor and a thermal flow velocity sensor, disposed on the same line parallel to the axis of the tool.

### CROSS-REFERENCE TO RELATED APPLICATION

This application claims priority to Russian Application No. 2016126377 filed Jul. 1, 2016, which is incorporated herein by reference in its entirety.

### BACKGROUND

The disclosure relates to the field of geophysics, namely, to performing a series of geophysical logging of vertical, inclined and horizontal oil, gas, gas-condensate and geothermal wells, in particular for measurement, indication, control and transmission of the physical parameters of producing and injection wells to the surface either in a real time via a wireline cable or a delayed transmission through storing data in an autonomous memory.

A wireline logging device is known for well monitoring horizontal wells during development and production stages (Patent RU 2442891), comprising a cylindrical housing, a lever centralizer aligning the tool along a well axis and having at least six levers and a fluid flow temperature sensor and thermal flow sensor located on the tool axis. Fluid phase composition sensors are located on the centralizer levers and distributed along the well bore circumference. An additional fluid phase composition sensor is located on the tool axis. At least one additional fluid flow temperature sensor and at least one additional thermal flow sensor are disposed on each lever and distributed along the well bore circumference and located on the same line with the phase composition sensors parallel to the tool axis. There is an additional upper lever centralizer in the tail part.

The disadvantage of the known tool is the narrow field of application due to limited functionality, since the tool provides measurements exclusively in the conditions of stratified flow typical of marginal horizontal wells.

### SUMMARY

The disclosure provides for increasing the information content of logging and efficiency of the tool, expanding functionality in conditions of a multiphase flow, including a stratified flow, in sub-vertical, inclined and horizontal wells.

The complex tool according to the disclosure comprises a cylindrical housing and at least two lever centralizers aligning the tool along a well axis. Each centralizer has at least three levers, as well as at least one fluid flow temperature sensor, at least one phase composition sensor and at least one thermal flow velocity sensor, all sensors being located on an axis of the tool. The tool comprises also at least three groups of sensors arranged to be distributed around a perimeter of the wellbore when the levers of at least one centralizer are being opened. Each group of the sensors comprises at least a fluid flow temperature sensor, a fluid phase composition sensor and a thermal flow velocity sensor, disposed on the same line parallel to the axis of the tool.

Any of the thermal fluid flow velocity sensors can operate in a constant, pulse or intermittent heating regime.

In accordance with one embodiment, the at least one fluid flow temperature sensor is combined with the fluid phase composition sensor.

According to another embodiment, the at least one thermal fluid flow velocity sensor is combined with the fluid phase composition sensor.

According to one more embodiment, the temperature sensor in at least one group of sensors is disposed first relatively to fluid flow direction.

### BRIEF DESCRIPTION OF DRAWINGS

The disclosure is explained by the drawings, in which

FIG. 1 shows an example of an embodiment of the complex tool with two opened centralizers, all sensors are located on one centralizer;

FIG. 2 shows a layout of the tool housing and one of possible radial positions of six groups of sensors;

FIG. 3a shows radial location of the sensors in accordance with one embodiment of the disclosure when the centralizer is in closed position,

FIG. 3b shows radial location of the sensors in accordance with the same embodiment of the disclosure when the centralizer is opened.

### DETAILED DESCRIPTION

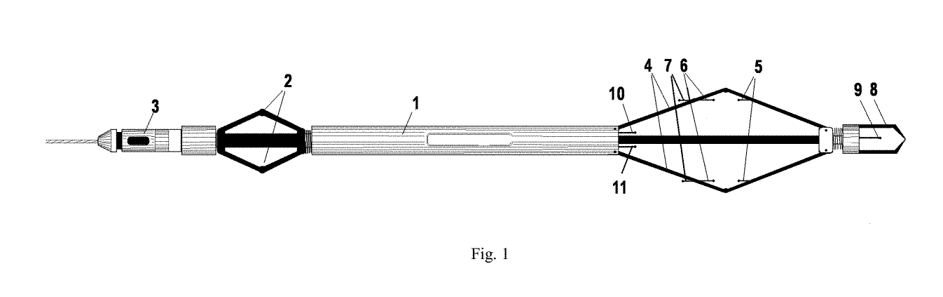

As shown in FIG. 1, the complex well tool is a cylindrical housing 1 in which any known sensors (for example, but not limited to, collar locator CL, gamma channel GC, pressure MN, passive multichannel sound level meter SLM, attitude determination sensors XYZ, electronics boards) can be disposed. An upper centralizer comprising at least three levers 2 may be located above the cylindrical body 1 in the tail section of the tool after a plug-and-socket cable terminal 3. A head centralizer having at least three levers 4 comprises at least three groups of sensors. In this example, each group of sensors is arranged on a lever and includes at least one temperature sensor 5, at least one phase composition sensor 6 and at least one thermal fluid flow velocity sensor 7. All sensors of the same group are disposed on the same line parallel to the tool axis. An axial temperature sensor 9 is mounted in a nose fairing 8, and an axial phase composition sensor 10 and an axial thermal fluid flow velocity sensor 11 are mounted in the tool housing. At least one of the temperature sensors (e.g., 5 or 9) can be combined with the thermal flow velocity sensor (e.g., 6 or 10) located respectively on the same line parallel to the axis of the tool.

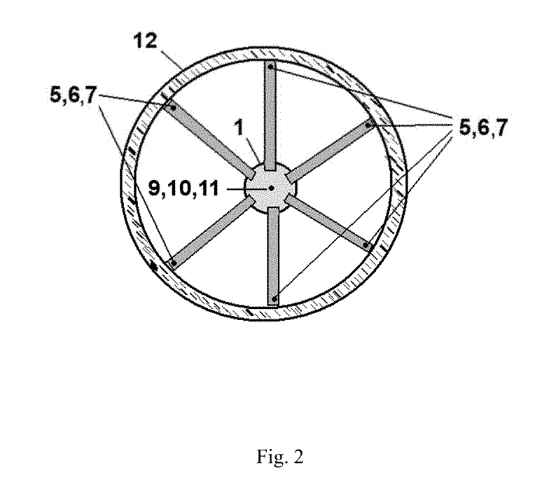

The spring-loaded levers 4 provide alignment of the tool body 1 along the axis of a subvertical, inclined and/or horizontal well 12 (FIG. 2) and uniform or non-uniform azimuthal distribution in the transverse cross-section of the well of at least three groups of sensors, each comprising at least one temperature sensor 5, at least one phase composition sensor 6 and at least one thermal fluid flow velocity sensor 7. In this case, the sensors 9, 10 and 11 are located on the axis of the well.

The levers 2 of the upper centralizer can also be equipped with groups of temperature sensors, phase composition sensors and thermal flow velocity sensors. All sensors of one group are located on the same line parallel to the tool axis. All the groups are distributed (for example, at an equal distance) along the cross-section of the wellbore, similar to the head lever centralizer.

For example, thermal anemometers can be used as the thermal fluid flow velocity sensors, including, but not limited to, thermal anemometers in constant heating power or constant overheating regime, with pulse, intermittent or continuous heating regime.

The presence of a phase composition sensor, a thermal flow velocity sensor and a fluid temperature sensor is necessary.

In accordance with one embodiment of the disclosure, at least one fluid flow temperature sensor can be combined with a fluid phase composition sensor. According to another embodiment, the at least one thermal fluid flow velocity sensor can be combined with a fluid phase composition sensor.

Any of the thermal fluid flow velocity sensors can operate in a constant, pulse or intermittent heating regime.

The thermal flow velocity sensors in different groups can operate in the same or in different heating regime.

All sensors of the same group, positioned relative to the tool axis, can be arranged on the levers of one, two or more different centralizers. If all groups of sensors are located on the levers of one centralizer, all groups of sensors can be located on the same circumference in the cross-section of the well and distributed evenly or unevenly therein. When positioned on two or three centralizers, the sensors of the same group can be located on the same line parallel to the tool axis, their mutual position on the line parallel to the tool axis may be selected randomly, but the temperature sensors should be preferably be placed first relatively to the fluid flow direction.

Each sensor or a group of sensors or all sensors can be arranged on one or more centralizer levers or on a special auxiliary mechanical device for distributing the sensors or groups of sensors over the cross-section of the wellbore in the selected azimuthal and radial positions. Thus, the number of sensors of any type or the number of groups of sensors can be equal to or greater than the number of levers of any centralizer.

The sensors are distributed in the azimuth direction evenly or unevenly. The radial position of each group of sensors is selected at a sufficient distance from the casing wall boundary pipes and from the levers and the housing of the tool to prevent excessive disturbance of fluid velocity and temperature by pipes or casing walls and tool components including the levers and the housing. This arrangement of the group of sensors is provided by auxiliary mechanic devices.

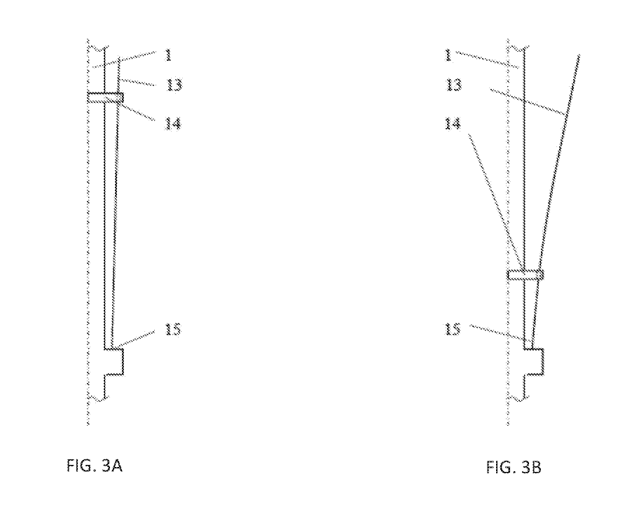

The principle of operation of one of the possible structures of such an auxiliary mechanical device is shown in FIG. 3. The auxiliary mechanical device may include, for example, flexible tubes 13 connecting the sensor and the tool housing 1 and a moving ring 14 with a number of holes (shown by a dashed line in the ring 14) equal to the number of sensors (but not necessarily equal to the number of levers). The minimum distance from the inner wall of the holes in the ring 14 to the tool 1 axis is greater than the distance from the attachment 15 of the flexible tube 13 to the tool axis. The ring 14 is mechanically connected to one or more moving elements of the centralizer.

When the centralizer is closed, the moving ring 14 is in the position shown in FIG. 3a, pressing together the flexible tubes 13 with the sensors 1 to the tool housing.

When the centralizer is opening, the moving ring 14 moves to the position shown in figure FIG. 3b, by deflecting the flexible tubes 13 from the tool housing by the desired distance.

The tool can be combined in one housing or in a measuring logging assembly with any known logging tool or tools and a sensor or sensors, for example, but not limited to, the tool may also include a fluid flow temperature sensor, a phase composition sensor and a thermal flow velocity sensor, all located along the axis of the tool.

The tool may be provided with at least one independent power source (for example, a battery) and at least one storage unit for providing autonomous data acquisition and storage.

One of possible embodiments of the complex well monitoring tool operates as follows.

After lowering the tool into the survey range and bringing it to the operating status, the centralizer's open and physical fields are recorded while the tool is being lowered. The tool position linking to the cross section and to the structure of the production casing is provided by any known linking methods (e.g., GC and CL, but not limited to these methods). Current pressure in the tool location point at the time of measurement is determined by a pressure sensor MN. The tool housing and active centralizer sensors' attitude determination is performed, but not limited to, relative to the gravitational field of the Earth using attitude determination sensor XYZ.

As shown in FIG. 2, the groups of temperature sensors 5, phase composition sensors 6 and thermal flow velocity sensors 7 record distribution of temperature, flow phase composition and flow velocity by the cross-section of the well 12, respectively, and the sensors 9, 10 and 11—along the tool axis. Attitude determination sensor, based on the Earth gravitation field, is linked to the position of one of the groups of sensors 5, 6 and 7 and provides the possibility of building temperature, phase composition and a local fluid flow velocity field along the cross-section of the well, using for example, but not limited to, the cubic spline interpolation method. A comprehensive analysis of all the recorded parameters, by taking into account the distribution of temperature fields, phase composition and local velocities by the flow cross-section, provides the possibility of unambiguous allocation of intervals of oil, gas or water inflow under conditions including stratified multiphase flow in the subvertical, inclined and horizontal wellbore. The location of thermal flow velocity sensors above the temperature sensors ensures that there is no distortion of the flow temperature field due to heat generation in the thermal fluid flow velocity sensors during recording of the parameters in a production well when lowering the tool. Location of the groups of temperature sensors, phase composition sensors and thermal flow velocity sensors on the same line parallel to the well axis ensures that an initial flow temperature, the fluid phase composition are taken into account and the local flow velocity is quantitatively estimated using the thermal flow velocity sensor.

The set of all measured parameters is continuously transmitted in real time to a surface recorder by a cable or is accumulated in the built-in memory of the tool. The measuring circuit and the tool as a whole are supplied with power via a cable or from autonomous power supply sources. Transportation of the tool along the subvertical, inclined and horizontal wellbores is carried out by standard devices intended for geophysical logging, including, but not limited to, a geophysical cable or a coiled tubing.

1. A complex tool for well monitoring, the tool comprising:
a cylindrical housing;
at least two lever centralizers aligning the tool along a well axis, each centralizer having at least three levers;
at least one fluid flow temperature sensor, at least one phase composition sensor and at least one thermal flow velocity sensor, located on a tool axis;
at least three groups of sensors distributed around a perimeter of the wellbore when the levers of at least one centralizer are opened, each group comprising at least one fluid flow temperature sensor, at least one fluid phase composition sensor and at least one thermal flow velocity sensor, disposed parallel to the tool axis.

2. The complex tool of claim 1, wherein the at least one fluid flow temperature sensor is combined with the fluid phase composition sensor.

3. The complex tool of claim 1, wherein the at least one thermal flow velocity sensor is combined with the fluid phase composition sensor.

4. The complex tool of claim 1, wherein the at least one thermal flow velocity sensor operates in continuous heating regime.

5. The complex tool of claim 1, wherein the at least one thermal flow velocity sensor operates in pulse heating regime.

6. The complex tool of claim 1, wherein the at least one thermal flow velocity sensor operates in intermittent heating regime.

7. The complex tool of claim 1, wherein the groups of the sensors are arranged on the levers of at least one centralizer.

8. The complex tool of claim 7, wherein the sensors of at least one group are located on the levers of different centralizers.

9. The complex tool of claim 1, wherein the temperature sensor in at least one group is disposed first relatively to the direction of fluid flow.
